(19) United States
(12) Patent Application Publication
(10) Pub. No.: US 2026/0224038 A1
(43) Pub. Date: Aug. 6, 2026

(54) BED-INTEGRATED MICROSTIMULATION SYNCHRONIZATION WITH AUDIO INPUT

(71) Applicant: Ergomotion, Inc., Goleta, CA (US)

(72) Inventors: Cort Corwin, Grand Haven, MI (US); Jordan Visser, Byron Center, MI (US); Richard Harris, Wyoming, MI (US); Eric Bryant, Nunica, MI (US)

(73) Assignee: Ergomotion, Inc., Goleta, CA (US)

(21) Appl. No.: 19/152,314

(22) PCT Filed: Jan. 29, 2024
(86) PCT No.: PCT/US2024/013294

§ 371 Date: Jul. 30, 2025

Related U.S. Application Data
(60) Provisional application No. 63/482,134, filed on Jan. 30, 2023.

(51) Int. Cl. A47C 21/00; A61M 21/00; A61M 21/02

(52) U.S. Cl. CPC A47C 21/003; A61M 21/02; A61M 2021/0022; A61M 2021/0027; A61M 2021/0044; A61M 2205/18

(57) ABSTRACT

A system for synchronizing bed-integrated microstimulation with audio inputs includes a bed, a controller in electronic communication with an audio output system for generating an auditory output and a microstimulation output system for generating a haptic output, where the controller is configured to synchronize the haptic output with the auditory output based on the audio input signal received by the controller. A method of operating an integrated sleeping system includes selecting a scene, the scene being defined by an audio input signal. The method includes playing an audio output sequence based on the audio input signal and generating a microstimulation haptic output sequence synchronized with the audio output sequence.

### CROSS-REFERENCE TO RELATED APPLICATION(S)

This application claims priority to and the benefit of U.S. Provisional Application No. 63/482,134, filed Jan. 30, 2023, the entire disclosure of which is incorporated by reference herein.

### TECHNICAL FIELD

This disclosure relates generally to the field of adjustable beds and more particularly relates to an integrated microstimulation synchronization system with audio source inputs for adjustable beds.

### BACKGROUND

Although humans require sleep, achieving the desired quality and quantity of sleep may be challenging. Variability between individual users in size, proportion, and preferences can make a bed design suited to one individual unsuitable for another. Adjustable beds are known to provide improved comfort to individuals by providing variable support firmness and positioning with adjustable panels, inflatable bladders, or the like. Manual modification of different settings can be tedious, time consuming, and detrimental to changing conditions that make optimization difficult.

Humans have sought to improve sleep quality through the use of noise making machines, massage devices and selective lighting conditions. Managing multiple different devices and operations can be cumbersome and distributive to a relaxing or meditative environment.

It is therefore desirable to provide an integrated bed system providing microstimulation haptic outputs synchronized with audio input to improve a user's relaxed state, and sleep quality and quantity.

### BRIEF DESCRIPTION OF THE DRAWINGS

These and other features and advantages of the present disclosure will be better understood by reference to the following detailed description of exemplary implementations when considered in connection with the accompanying drawings wherein:

FIG. 1 is a schematic view of an integrated system for microstimulation and synchronization with audio input in a first configuration.

FIG. 2 is a schematic view of an integrated system for microstimulation and synchronization with audio input in a second configuration.

FIG. 3 is a schematic view of an integrated system for microstimulation and synchronization with audio input in a third configuration.

FIG. 4 is a schematic flow chart of method of operating an integrated system for microstimulation and synchronization with audio input in a first configuration.

FIGS. 5A-5D illustrate a series of state transitions in executing an immersive scene using an integrated system for microstimulation and synchronization with audio input.

FIGS. 6A-6B illustrate state diagrams for a controller of an integrated system for microstimulation and synchronization with audio input in an automatic mode of operation and in a manual mode of operation, respectively.

### DETAILED DESCRIPTION

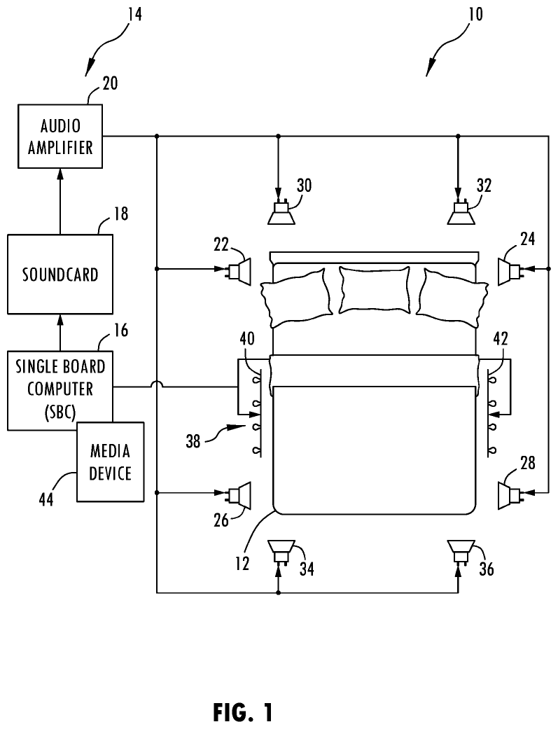

Embodiments shown in the drawings and described herein provide an integrated system for synchronizing stimulus to a user. Referring to the drawings, FIG. 1 illustrates an exemplary configuration of a system 10 for synchronizing an auditory output with a haptic output based on an input signal, such as an audio signal received by the system. The system 10 may further synchronize a visual output and a position of the user based on the input signal.

The system 10 operates by receiving an input signal, for example, representing an audio signal. The audio signal, when played as an audio track, may include a music track, nature sounds, spoken word, white noise, or the like. The system 10 analyzes the audio signal and transcribes the audio signal into a haptic signal to generate a haptic track to run in parallel with the audio track. By matching the timing of different amplitudes and frequencies in the audio track with corresponding amplitudes and frequencies in the haptic track.

Synchronization across the auditory and tactile senses can enhance brain entrainment, which is the body's ability to mimic or mirror external stimulus, such as wither respiratory rate or heart rate. In this way, an audio track of waves crashing or other pulsatile sequence with increasing interval over time can be used to slow a user's breathing and heartbeat to aid in mental and muscle relaxation, or meditation, improved circulation and memory retention. Further brain entrainment may be achieved by further synchronizing a lighting track with the auditory and haptic tracks. The 2 system 10 may further generate a lighting track to modulate the color or brightness or both of a lighting system or lighting device with the frequency and amplitudes of the auditory and haptic tracks.

As illustrated in FIG. 1, the system 10 includes a user support, such as bed 12. The bed 12 may a flat foundation or frame supporting a traditional coil spring or foam mattress. The bed 12 may include an adjustable bed frame configured to place the user in a position to improve reception of the synchronized auditory, tactile and lighting stimulus. For example, the adjustable bed frame may place the user in a zero gravity position, where the head and knees are raised slightly above the heart and the legs and torso are arranged at a roughly 120° angle. Starting from the end of the bed 12 near the user's feet, the bed 12 may include a foot support portion defining a first zone of the bed 12. The bed 12 may include a thigh support portion defining a second zone of the bed 12. The bed 12 may include a seat portion defining a third zone of the bed 12. And terminating at the end the bed 12 near the user's head, the bed 12 may include a head support portion defining a fourth zone of the bed 12. Adjustable bed frames including multiple support portions, and actuation systems for the same, are known, for example, from U.S. Pat. No. 8,683,629, entitled Articulating Bed With Lumbar And Head Adjustment, having a common assignee with the present application, the entire disclosure of which is incorporated by reference herein.

The system 10 includes a controller 14 for providing signal processing and electronic communication between and among the devices comprising the system 10. The controller 14 may include a single board computer (SBC) 16, mini-PC, or the like. A typical single board computer includes a microprocessor, memory device(s), data storage, and other input/output devices or interfaces. As will be described in further detail below, the controller 14 may use remote devices to provide user input and/or output. For example, the controller 14 may communicate with a smartphone device, smart home hub device or the like. The controller 14 may include or may be in communication with one or more sensors to sense a condition of the environment in which the system is used, or sensors to sense a condition of a user. The sensors may include temperature sensors, motion sensors, including accelerometers, gyroscopes, optical sensors or the like. Sensors may include heartbeat sensors, respiratory rate sensors, brain wave sensors and others. The SBC 16 may include or may be in communication with an audio processor, such as a sound card 18, or the like, as a dedicated processing device for processing and communicating the audio signal to the audio devices. The SBC 16 may also include or may be in communication with an audio amplifier 20 to further modulate the volume or intensity of the audio signals.

The audio amplifier 20, where present, or the SBC 16, may be in communication with one or more audio devices, such as speakers, and one or more haptic devices, such as vibratory transducers. As illustrated in FIG. 1, the audio amplifier distributes the audio and haptic tracks to four speakers 22, 24, 26, 28, and four vibratory transducers 30, 32, 34, 36, but this is not intended to be limiting and other numbers of speakers or vibratory transducer may be used. The speakers 22, 24, 26, 28 may be conventional speakers capable of producing sounds within the human auditory range of about 20 hertz (Hz) to about 20,000 Hz. Alternatively, each speaker 22, 24, 26, 28 may be a combination of multiple specialized devices capable of producing a narrower range of frequencies, such as subwoofers, woofers, midrange and tweeter devices. The vibratory transducer 30, 32, 34, 36 are capable of producing lower frequency vibrations below the threshold of human hearing but which may be felt and experienced by a user.

The vibratory transducers 30, 32, 34, 36 may be supported on a semi-rigid portion of a user support, such as a support panel of an articulating bed or reclining chair so that the compliance of the semi-rigid portion aids in transmitting the low-frequency vibrations through any spring, foam, or other mattress or cushion positioned between the user and the vibratory transducer. Depending on the power capability of the vibratory transducer and the relative dimensions and proportions of the user support components, the material selection, geometry, level of rigidity or compliance may be optimized for the vibration transmission. In one example, the semi-rigid portion may be formed of a fiber-reinforced polymer sheet. Variable levels of rigidity and compliance across the semi-rigid portion may be obtained by the placement of cutouts or increased or reduced thickness in certain areas of the semi-rigid portion.

The audio amplifier 20 may be arranged to communicate to each respective speaker and vibratory transducer on its own distinct channel or may communicate to multiples of the speakers or the vibratory transducers together, depending on the number and complexity of the speakers and vibratory transducers employed in the system, depending on the complexity of the input signal, or depending on a user's selections or preferences. In the illustrated configuration, the speakers and vibratory transducers may be independently addressed based on their left/right and head/foot positioning. Specifically, speaker 22 and vibratory transducer 30 are arranged proximate the left side head end of the bed 12 and the speaker 24 and vibratory transducer 32 are proximate the right side head end of the bed 12. Speakers 26, 28 and vibratory transducers 34, 36 are similarly paired at the left and right sides of the foot end of the bed 12. By varying the volume and intensity of the output of the system 12 from the left to the right and from the foot to the head can produce a three-dimensional effect of moving the stimulus around or across the user resting on the bed 12.

The system 10 illustrated in FIG. 1 includes a lighting system 38 in communication with the controller 14. The lighting system 38 includes a first LED strip 40 arranged along a left side of the bed 12 and a second LED strip 42 along a right side of the bed 12. The LED strips 40, 42 may include a plurality of individually addressable light emitting diodes capable of producing light across a range of dimmable intensities and colors. The LED strips 40, 42 may extend the entire length of the side of the bed from the head end to the foot end. In other alternatives, the LED strips 40, 42 may each respectively comprise a plurality of LED strip segments disposed, for example, on each of the head support section, the seat section, the thigh support portion, and the foot support portion respectively. In further alternatives, the lighting system 38 may include other lighting technologies, such as incandescent, fluorescent, and the like. The lighting system 38 may be arranged to synchronize the illumination color, intensity, and patterns to the audio or haptic tracks to complement the stimulus provided to the user.

The system 10 may include or may be in communication with a media device 44 configured to provide or to generate an audio input to drive the synchronized auditory, haptic, and visual stimulus to the user. As described above, the SBC 16 may include a memory device that can store audio tracks, such as a .wav or .mp3 audio files. In other alternatives, the SBC 16 may include communication devices, such as a radio or modem configured to communicate over WiFi, Bluetooth, or other wired or wireless communication protocols to transfer or receive audio tracks from an external or remote device, such as media device 44. The SBC 16 may communicate over the internet with a cloud-based server. The SBC 16 may communicate with a mobile device, such as a smartphone, as a gateway device to access a cloud-based server or service that provide audio tracks. The mobile device may be a smart home hub integration device.

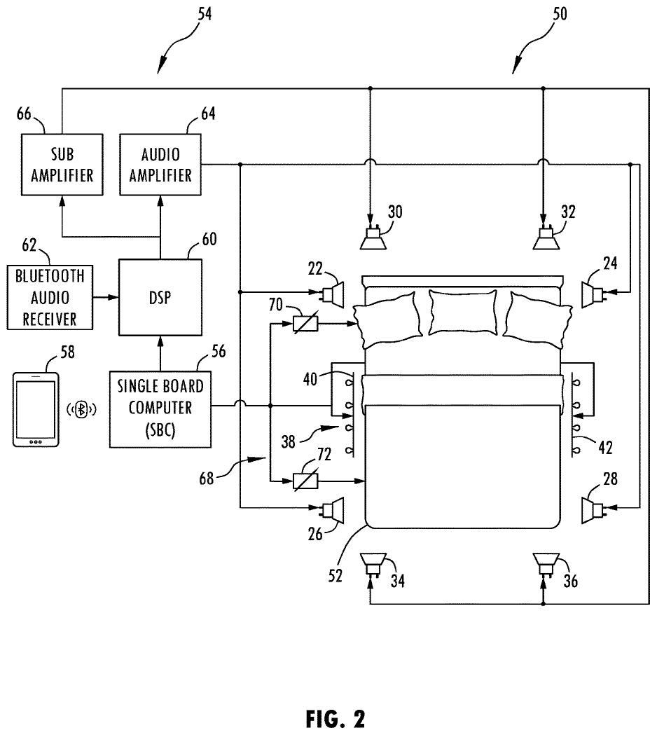

Referring to FIG. 2, a second exemplary implementation of a system 50 is illustrated. The system 50 includes an adjustable bed 52 provided with a matrix array of speakers 22, 24, 26, 28 and vibratory transducers 30, 32, 34, 36 arranged similarly to the implementation illustrated in FIG. 1. The system 50 includes a controller 54 comprising a SBC 56 arranged to communicate via Bluetooth with a user device 58 through a digital signal processor (DSP) 60 having a Bluetooth audio receiver 62. The DSP 60 facilitates the communication of the audio tracks to the audio amplifier 64 that in turn communicates to the speakers 22, 24, 26, 28. The DSP 60 also communicates a haptic track based on the audio track to a sub amplifier 66 that in turn communicates with the vibratory transducers 30, 32, 34, 36. The operation of the system 50 is similar to the system 10 but with separate amplifiers for the audio track and the haptic track. The addition of a separate amplifier may provide greater control of additional channels separating the audio from the haptic tracks. However, the additional amplifier adds cost and complexity to the system.

The system 50 includes a lighting system 38 and further includes an articulation system 68 in electronic communication with controller 54. The articulation system 68 includes one or more actuators to adjust the position of the articulating support portions of the adjustable bed 52. The articulation system may include a head actuator 70 and one or more foot actuators 72. The thigh support portion and the foot support portion may be linked to one another such that a single actuator works to articulate the thigh and foot support portions in cooperation. The controller 54 may control the operation of the articulation system 58 to transition the adjustable bed 52 from a first position, such as a flat position, to a second position, such as a zero-G position, a reclining position with the head portion elevated but the foot and thigh portions flat, or a declining position with the foot and thigh portions elevated and the head portion flat. The controller 54 may operate the articulation system 68 automatically in synchronization with a start of a particular audio track. Alternatively, controller 54 may operate the articulation system 68 manually in response to a user input or selection.

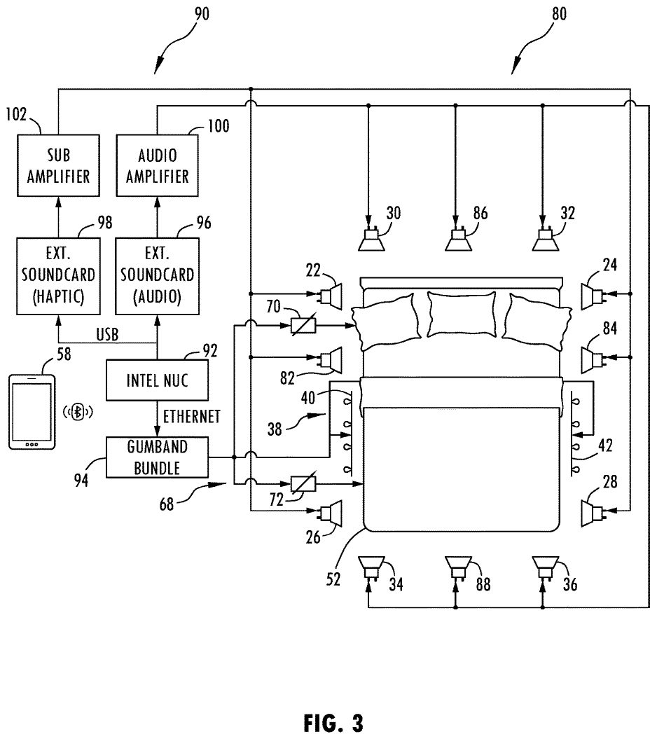

Referring to FIG. 3, a third exemplary implementation of a system 80 is illustrated. The system 80 includes an adjustable bed 52 provided with a matrix array of speakers 22, 24, 26, 28, 82, 84, and vibratory transducers 30, 32, 34, 36, 86, 88. Similar to the preceding systems 10, 50, the array of speakers and vibratory transducers are distributed about the adjustable bed 52 from the left side to the right side and from the head end to the foot end. Unlike the above systems 10, 50, system 80 includes an additional pair middle speakers 82, 84 disposed, for example, at the seat portion of the adjustable bed, and an additional pair of vibratory transducers 86, 88 are provided and may be positioned proximate the middle speakers 82, 84 at the seat portion, or may be arranged centrally along the head support portion and the foot support portion respectively. The additional pairs of speakers 82, 84 and vibratory transducers 86, 88 allows the system 80 to provide greater granularity in the stimulus provided to the user in three-dimensional variability.

The system 80 also includes a controller 90. The controller 90 includes a mini PC 92, such as an Intel NUC. The controller 90 includes an input/output microcontroller 94, such as is commercially available as the Bundle produced by Gumband. The mini PC 92 may be connected to the input/output microcontroller 94 via an Ethernet connection or other suitable wired or wireless communications protocol. The input/output microcontroller may provide an interface to an articulation system 68 and a lighting system 38, and other input or output devices. The mini PC 92 may include or may be in communication, e.g., via USB, with an external soundcard for the audio channels 96 and an external soundcard for haptic channels 98. The external soundcards 96, 98 may be in communication with respective audio and haptic amplifiers 100, 102 before the audio and haptic tracks are output to the array of speakers 22, 24, 26, 28, 92, 84 and vibratory transducers 30, 32, 34, 36, 38, 86, 88.

The systems 10, 50, 80, described above may be integrated into a structure of the bed, such as mounted to a frame component or support panel of a bed 12, 52. In other alternatives, the system 10, 50, 80 may be selectively or permanently coupled to a user support other than a bed. The user support may be chair, such as a reclining chair, a massage table, a seat or bed in a conveyance such as a train, plane, cruise ship, yacht, or the like. The system components may be permanently or removably secured to the user support. The system components may be integrated into the user support, such as, for example, by disposing a speaker or vibratory transducer into a headboard, or support panel, or chair back or seat. The controller of the system may be disposed in an accessible location while using the user support to provide easy interface with the system to input selections or to facilitate installing updates to the system. The system may also be configured for over-the-air updates having suitable WiFi or cellular connectivity devices.

The user support may be provided with multiple attachment points for repositioning the speakers and/or the vibratory transducers to provide for a more tailored user experiences. For example, where the user support is an adjustable bed having separate support portions for the head, seat, thigh and foot, each support portion may include one or more mounting locations to which the speakers or vibratory transducers may be secured. The mounting locations may include a slot, bracket, snap-in, or other hardware design to provide easy assembly, disassembly, and relocation of the speakers and vibratory transducers. In one example, a taller user may place the speakers and vibratory transducers spaces apart a greater distance to provide a more immersive experiences and a shorter user may place the speakers and vibratory transducers closer together to provide a more intensive experience.

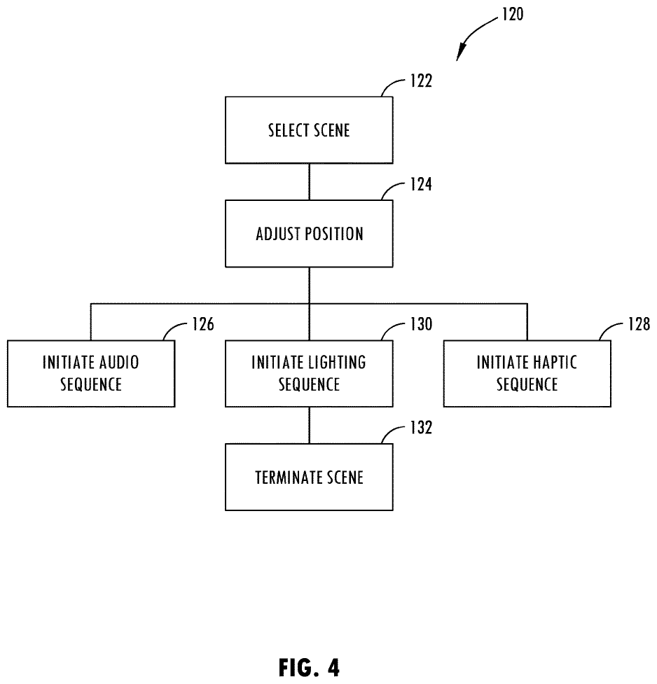

Referring to FIG. 4, a method 120 of operating a system 10, 50, 80 is illustrated. The method 120 includes a first step of selecting a scene 122. The step of selecting a scene 122 may be performed by a user on an input device of the system 10, 50, 80 directly, such as by a keypad, button selection, or other input of the controller. Alternatively, the step of selecting a scene 122 may be performed by the user on a remote device in electronic communication with the controller of the systems. The remote device may be a user's smartphone device paired to the controller via Bluetooth. In further alternative, the selection of a scene may performed by setting a future time for a scene to be executed.

The scene to be selected may be stored on a memory device of the system controller, the user's smartphone device, or on a cloud server accessible by the system controller, or user's smartphone device, or other computing device. The smartphone or other user device may include a meditation app that provides guided meditation audio tracks. The scene selection may comprise a predetermined set of scenes, such as a ‘pre-sleep’ scene, a ‘get back to sleep’ scene, an ‘energize’ scene, and ‘relax’ scene. Each scene selection may comprises one or more audio tracks to execute or for selection by the user. The audio tracks of each of the scenes may be characterized according to their objective. For example, an audio track in the ‘pre-sleep’ scene may include mid-tempo audio sequence that decreases over a preset or selected time period to a low-tempo to aid the user in falling asleep with little to no decrease in volume/amplitude. Similarly, a ‘get back to sleep’ scene may start with a low-tempo and decrease in both tempo and volume/amplitude to assist a restful user in falling back asleep after being unexpectedly awoken. An ‘energize’ scene may comprise an audio track with increasing tempo and volume/amplitude to increase the respiration rate and heartbeat of the user. A ‘relax’ scene may have a substantially constant tempo and volume/amplitude. The user may also select a scene to be developed on-the-fly by the controller by providing an audio track input from, for example, a user's music library.

Once a scene is selected in step 122, the method 120 includes, optionally, adjusting the position of the user support at step 124. Where the user support is an adjustable bed including an articulation system, the method may include adjusting, by the controller, a position of support portions of the adjustable bed. The adjustment may move the user from a flat position to a zero-G position with head support portions and leg support portions being raised. Other position adjustments may be dictated by the user support on which the system is implemented, or by the scene being selected. For example, the selection of an ‘energize’ scene may transition a flat position to an elevated head position. The selection of a ‘get back to sleep’ scene may not involve any position change. The controller may also allow the user to associate specific position changes with specific scene selections. The controller may also develop a position change on-the-fly based on an audio track input from a user's music library by analyzing the tempo or volume changes within the audio track and comparing those parameters with, for example, audio tracks associated with ‘get to sleep,’ ‘energize,’ or ‘relax’ scenes and associating similar selections and preferences to the audio track input.

Once the user is in the adjusted position in step 124, the method 120 includes, in synchronization, initiating the audio sequence at step 126, initiating the haptic sequence at step 128 and, optionally, initiating the lighting sequence at step 130. As described above, the audio track, the haptic track and the lighting track are executed by the system in synchronization through the speakers, the vibratory transducers, and lighting systems. The controller, or a computing device in communication with the controller, may store an audio track, haptic track, and lighting track to drive the selected scene experience in synchronization. Where the scene selection is based on an audio track input, the controller generates the corresponding haptic track and lighting track on-the-fly to provide a customized experience. During the scene, the system may be configured to respond to user input to adjust or modify the scene parameters. In one example, the controller may include or may be in communication with a microphone and suitable voice processing software for receiving voice control commands to increase or decrease scene intensity. Voice control may also be used for other functions, such as system initialization, scene selection, scene termination or other operations. The controller may include or may be in communication with optical sensors, such as a camera, and suitable video process software for receiving gesture control commands. A stereoscopic camera array may allow the system to receive three-dimensional gesture control. Gesture control commands could include a wave toward the head end for increasing scene intensity, a wave toward the foot end for decreasing scene intensity, and a side-to-side wave for scene termination. Other suitable control means, such as touch control, pressure sensitive surfaces, and keypads, sliders, switches and the like may be integrated with the user support for providing user input to the system.

At the end of the synchronized experience driven by the audio track, the haptic track and the lighting track, the scene terminates at step 132. At the termination of the scene, the system may enter a low power, standby mode awaiting the next scene selection. Alternatively, the controller may terminate the scene and transition to a following scene based on a user-selected, or predetermined playlist of scenes. Scenes may also be scheduled to execute at set times to assist a user in falling asleep or waking up at a consistent time.

The scene selection step 122 and scene termination step 132 may be determined by the controller in a closed loop operation based on physiological parameters of the user monitored and analyzed by the controller. In one example, a sleep tracker measures a user's heart rate, respiratory rate, and heart rate variability, among other parameters, and communicates the data representing these parameters to the controller. The controller may be configured to automatically select a scene based on the physiological parameters meeting certain thresholds or trends evidencing a user waking from a sleep during a sleep period. For example, the controller may select the ‘get back to sleep’ scene when the heart rate variability indicates the user is waking in the middle of a sleep period. Similarly, the step 132 of scene termination may be tied to monitored physiological parameters. Executing a ‘relax’ scene, the controller may receive data representing a user's heart rate and respiratory rate. The controller may record a starting value and may be configured to trigger a scene termination upon a pre-selected decrease in the heart rate and respiratory rate. Other parameter monitoring may be incorporated in addition or in the alternative. For example, wireless brain wave monitoring, body movement, position or indications of restlessness or other parameters may be used by the controller to trigger scene selections or scene terminations.

Where a bed is arranged to accommodate multiple users, the controller may integrate multiple sensing and logical operations to target a scene's stimulation to one user while excluding the other user or users. Sensors in communication with the controller may allow the controller to determine a position on the bed of the user whose physiological parameters are triggering the scene initiation, and to determine a position on the bed of the other user or users. With the individually addressed speakers and vibratory transducers, the volume and intensity of the audio and haptic tracks respectively may be controlled to stimulate one user and insulate the other user from the scene's stimulation. In one example, a scene may be executed to stimulate a user positioned on a left side of the bed by using only the speakers and vibratory transducers arranged on a left side of the bed. Speakers and vibratory transducers arranged in a middle position or along a right side of the bed may be idle during the scene or may be operated at a diminished intensity or modified setting. In one example, the speakers and vibratory transducers arranged between the users may operate at a lower volume and intensity, while the speakers and vibratory transducers along the right side of the bed operate at a phase-shifted or inverse amplitude to negate the effects of the scene for the second user. The scene intensity may be dependent on the physiological parameters of the other user or users so that if the controller determines that the scene execution is disturbing the restfulness of the other user or users, the intensity may be automatically lessened.

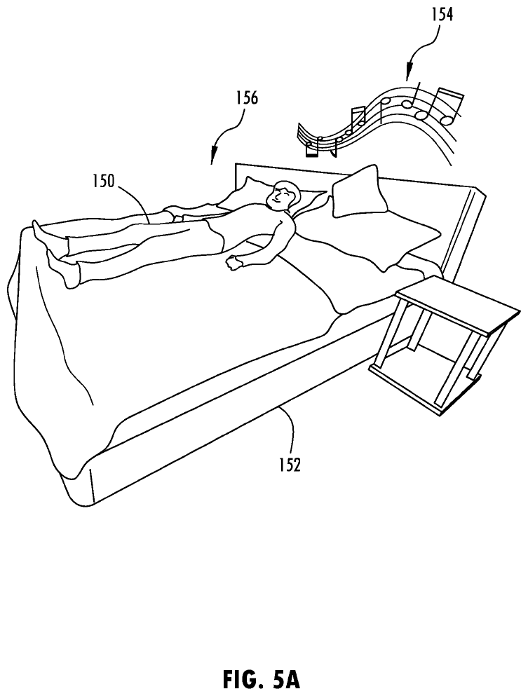

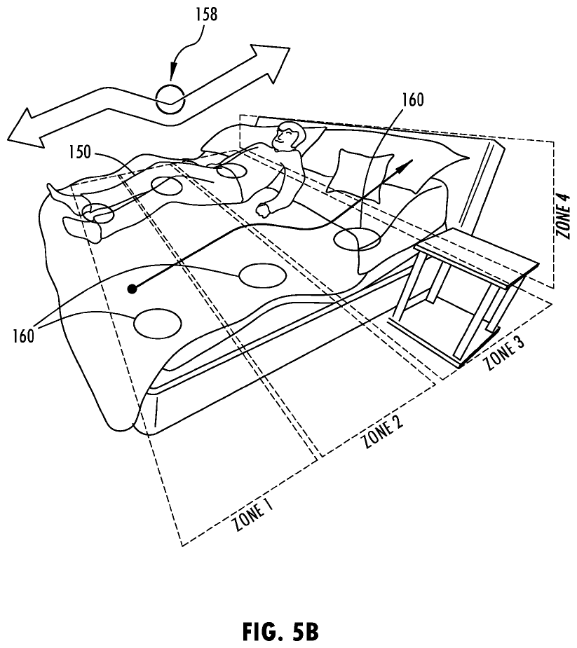

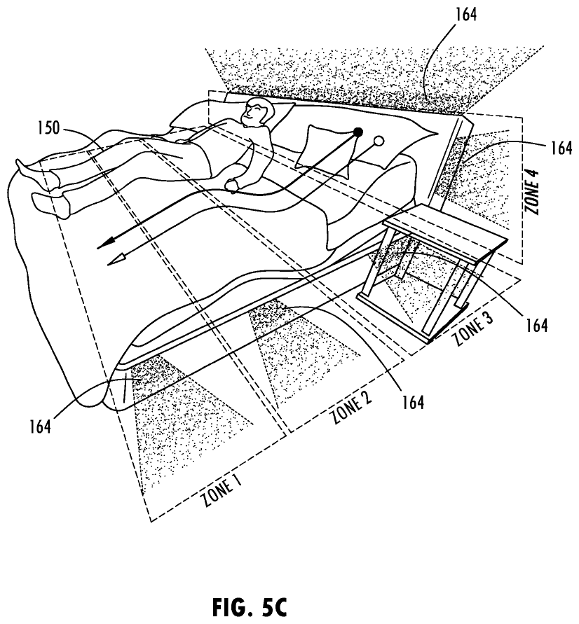

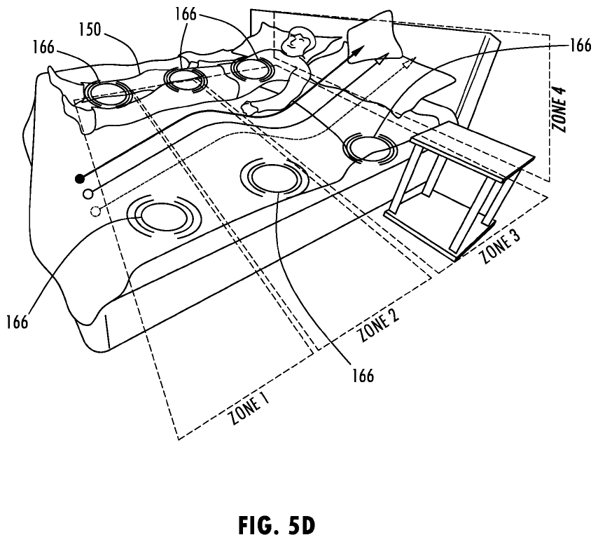

Referring to FIGS. 5A-5D, an example execution of the above described systems and method is illustrated. In a first stage, a user 150 lays in a bed 152 and a scene is selected either manually by the user 150, or automatically by the controller integrated into the bed 152 based on a predetermined schedule or based on monitored physiological parameters. The scene selection dictates a bed position track, an audio track, a haptic track and a lighting track to be executed by the system for immersing the user 150. In a first stage of the scene, a sound bar integrated in the bed's headboard plays background music 154 and the bed 152 moves from a flat position 156 to a zero-G position 158 (shown in FIG. 5B). Upon reaching the zero-G position 158, a matrix array of speakers 160 distributed around the bed 152 begin in zone 1 at the foot end of the bed 152 with sounds of a wave crashing on a shore. The sounds begin at a low volume and transition up the length of the bed 152 to simulate to the user the sound perception of the wave movement from the user's feet to the user's head in zone 4. The wave sounds may recede similarly from zone 4 to zone 1 as a wave would be experienced by a user laying on a shoreline. LED light strips 164 distributed in the zones join the transitioning wave sounds creating the effect of a wave of light to mirror the wave of sounds from the speakers 160. In concert with the sounds and the lights, microstimulation from vibratory transducers 166 distributed in the four zones create the feeling of being in the wave of light and sounds.

The scene may be arranged to introduce sound, light and microstimulation simultaneously or else the separate sounds, light and haptic tracks may be arranged to include delays so that the sensory stimulations are introduced one at a time. The selection of delay durations may be selectable by the user. Similarly, the intensity of one or more of the tracks may be selectable by the user either individually or collectively to effective “turn up” or “turn down” the experience. The intensity selection may be provided as a slider on a graphical user interface presented to the user, for example, on a smartphone device or a smart home hub device.

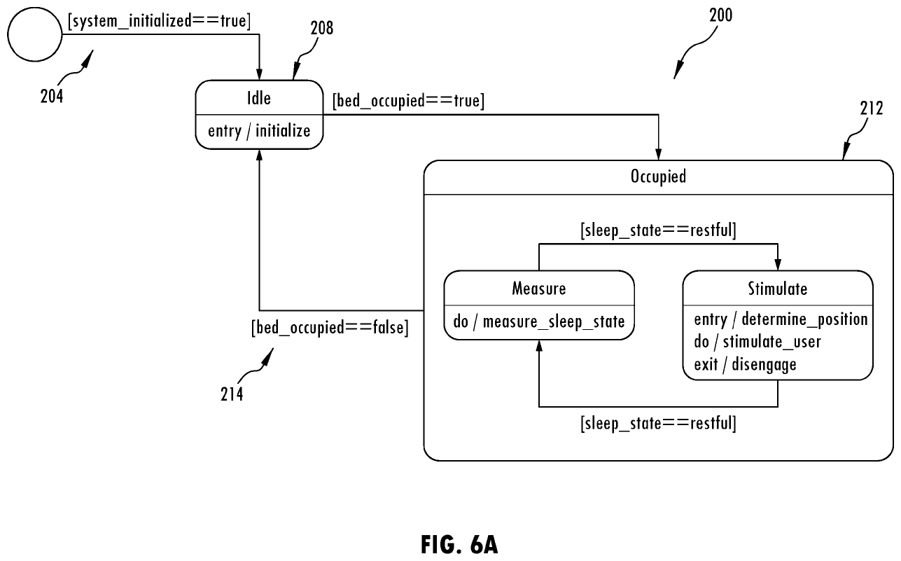

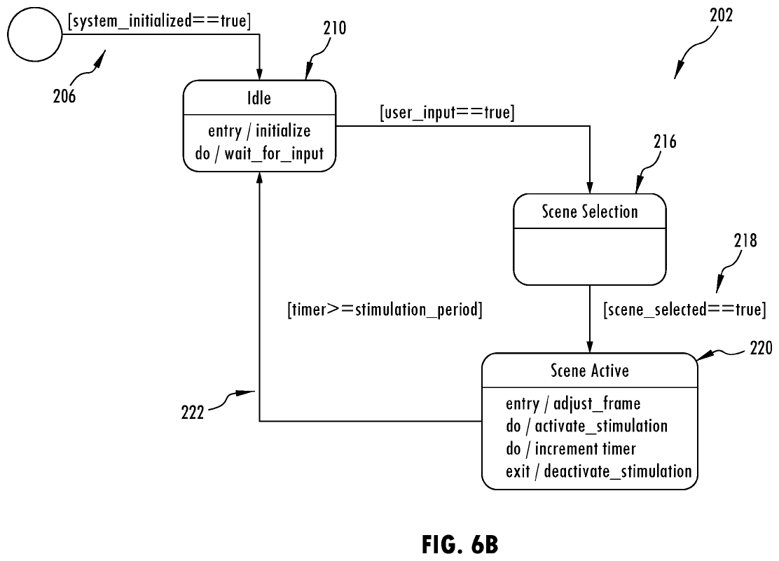

FIGS. 6A-6B further detail modes 200, 202 of controlling the above described systems either automatically or manually based on user input, where the system is implemented with a bed as the user support, but which can be implemented with other forms of user support. At an initial phase 204, 206 the system is initialized, enters an idle state 208, 210, and awaits further control input or commands. When in an automatic mode of control (FIG. 6A), the system monitors whether the bed is occupied, for example, with a weight sensor, motion sensor, optical sensor or the like. While the system determines the bed is occupied 212, the system monitors the user state, such as their restfulness, based on a user's pose, motion, or other parameter via, for example, motion sensor, optical sensor, or the like. If the system determines the user is not restful 214, the system may be configured to automatically initiate a scene, such as a ‘get back to sleep’ scene. The system may then continue to monitor the user's restfulness state, and may be configured to initiate a scene using different parameters if within a predetermined amount of time from a prior scene. For example, the system may initiate a different scene selection, such as a ‘relax’ scene or ‘get to sleep’ scene, or may initiate the same scene using a different intensity value. Sequential scene operations may be selectively programmed by the user or may follow a default program determined empirically to provide the highest improvement in restfulness. While the user maintains a level of restfulness, the system continues to monitor the user. The system may initiate a scene to rouse the user from sleep, similar to an alarm clock. When the system determines that the bed is not occupied 214, the system returns to an idle state.

The user may manually operate the system. From an idle state 210, the user may provide an input selection 21, for example, via a smartphone Bluetooth-connected to the system or a smart home hub device to initiate a scene. The input may be in the form of a scene selection 216. The user input may be in the form of an audio track, from which the system generates the scene on-the-fly. Other forms of user input are contemplated to be within the scope of the present disclosure. With the scene selection 218, the system executes the synchronization of the position adjustment, if needed, audio track, haptic track, and lighting track as defined for the scene 220. The scene may be defined, for example, by a duration of the audio track and conclude once the total duration time elapses. Other terminations may be triggered based on a sensor input of the user's sleep state or otherwise. At the termination of the scene, the audio track, haptic track and lighting track are stopped and the position adjustment may be returned to the prior position, or a default position. After the scene has been deactivated, the system returns to an idle state 222.

As described above, the systems 10, 50 and 80 can be employed in alternative implementations to achieve a desired output selected by a user. In a first example, the scene selection (as in operation 122 shown in FIG. 4) may mimic physiological sensor data, such as by modulating one or more of a sound intensity, vibratory intensity or lighting intensity with a breathing rate. The system 10, 50, 80 can be employed to obtain a target breathing rate to call a user and aid transition to sleep, for example, by decreasing a breathing rate to a target breathing rate that is different from a user's sensed breathing rate. Scene selection may also be selected to be time triggered such as for an alarm clock function to an aid in waking and transitioning away from sleep or as a reverse alarm clock for transitioning to sleep.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature; may be achieved with the two components (electrical or mechanical) and any additional intermediate members being integrally formed as a single unitary body with one another or with the two components; and may be permanent in nature or may be removable or releasable in nature, unless otherwise stated. Similarly, the terms “supported,” “joined,” “mounted,” in all their forms, should be understood similarly to include directly or indirectly, permanently or impermanently, rigidly or resiliently, unless stated otherwise.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional implementations that also incorporate the recited features. Furthermore, the terms “first,” “second,” and the like, as used herein do not denote any order, quantity, or importance, but rather are used to denote element from another.

Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired 13 result. For example, the terms “approximately,” “about,” and “substantially” may refer to an amount that is within less than 5% of, within less than 1% of, within less than 0.1% of, and within less than 0.01% of a stated amount.

Further, it should be understood that any directions or reference frames in the preceding description are merely relative directions or movements. For example, the terms “upper,” “lower,” “right,” “left,” “rear,” “front,” “vertical,” “horizontal,” and derivatives thereof shall relate to the orientation shown in FIG. 1. However, it is to be understood that various alternative orientations may be provided, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Changes and modifications in the specifically described embodiments may be carried out without departing from the principles of the present invention, which is intended to be limited only by the scope of the appended claims as interpreted according to the principles of patent law. The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

1. A system for synchronizing bed-integrated microstimulation with audio inputs, the system comprising:
a bed;
a controller, the controller comprising a computing device supported on the bed foundation;
an audio output system for generating an auditory output, the audio output system in electronic communication with the controller and supported on the bed foundation;
a microstimulation output system for generating a haptic output, the microstimulation output system in electronic communication with the controller and supported on the bed foundation;
wherein the controller is configured to synchronize the haptic output with the auditory output based on an audio input signal received by the controller.

2. The system of claim 1 wherein the bed foundation comprises a bed frame and a mattress.

3. The system of claim 2, wherein the bed frame is an articulating bed frame comprising a support frame, and an articulation system.

4. The system of claim 3, wherein the articulation system comprises a head support portion, a seat portion, a thigh support portion, a foot support section, a first actuator arranged to articulate the head support portion relative to the seat portion and a second actuator arranged to articulate one of the thigh support portion, the first support portion or both the thigh support portion and the foot support portion relative to the seat portion.

5. The system of claim 4, wherein the audio output system comprises a speaker array including a first speaker supported on a head support portion, and a second speaker supported on a foot support section.

6. The system of claim 5, wherein the microstimulation output system comprises a vibratory transducer array including a first vibratory transducer supported on a head support portion, and a second vibratory transducer supported on a foot section.

7. The system of claim 6, wherein controller is configured to synchronize an actuation of the articulation system to the haptic output, the auditory output or both the haptic output and the auditory output.

8. The system of claim 6, wherein at least one of the head support portion and the foot section comprises a semi-rigid panel that comprises a fiber-reinforced polymer sheet.

9. (canceled)

10. The system of claim 9, wherein the fiber-reinforced polymer sheep comprises a cutout portion, an increased thickness portion, a decreased thickness portions, or combinations thereof.

11. The system of claim 1, wherein the controller comprises a single board computer and an audio amplifier in electronic communication with the single board computer, the audio amplifier in electronic communication with the audio output system and a microstimulation system.

12. The system of claim 1, wherein the audio output system comprises a speaker array arranged in a spaced configuration supported on the bed.

13. The system of claim 1, wherein the microstimulation system comprises a vibratory transducer arranged in a spaced configuration supported on the bed.

14. The system of claim 1, wherein an amplitude and frequency of the haptic output corresponds to a volume and frequency of the auditory output.

15. The system of claim 1 further comprising a lighting system, and wherein the controller is configured to synchronize a lighting output with the haptic output and auditory output based on an audio input signal received by the controller.

16. The system of claim 15, wherein the lighting system comprises a first lighting array arranged on a first side of the bed and a second lighting array arranged on a second side of the bed, and wherein a brightness and color of the lighting output corresponds to an amplitude and frequency of the haptic output and/or a volume and frequency of the auditory output.

17. (canceled)

18. A method of operating an integrated sleeping system comprising:
selecting a scene, the scene comprising an audio signal input;
playing an audio output sequence based on the audio signal input; and
generating a microstimulation haptic output sequence synchronized with the audio output sequence.

19. The method of claim 18, further comprising adjusting a position of a user support based on the audio signal input.

20. The method of claim 18, further comprising illuminating a lighting sequence synchronized with the audio output sequence.

21. The method of claim 18, wherein the step of selecting a scene is performed automatically based on a sensed physiological parameter of a user.

22. The method of claim 21, wherein the steps of playing an audio output sequence and generating a microstimulation haptic output sequence are terminated based on a change in a monitored physiological parameter of the user.
